SEQUENCE LISTING (1 sequence)

SEQ ID NO: 1 - protein, 5 aa, Artificial Sequence
  MOD_RES (2): Variable amino acid
  Description of Artificial Sequence Synthetic peptide
FXGLM

(19) United States
(12) Patent Application Publication
(10) Pub. No.: US 2007/0048225 A1
(43) Pub. Date: Mar. 1, 2007

(54) NK3 receptor in vivo assay

(76) Inventor: Will Spooren, Franken (FR)

Correspondence Address: HOFFMANN-LA ROCHE INC.;PATENT LAW DEPARTMENT, 340 KINGSLAND STREET, NUTLEY, NJ, 07110, US

(21) Appl. No.: 11/504,241

(22) Filed: Aug. 14, 2006

(30) Foreign Application Priority Data
Aug. 29, 2005 (EP) 05107892.1

(51) Int. Cl. A61K 49/00; A61K 38/08

(52) U.S. Cl. 424/9.2; 514/17

(57) ABSTRACT

The present invention relates to a method of assessing the in vivo potency of a NK3 receptor antagonist comprising: a) the non-human animal with the NK3 receptor antagonist of interest, b) treating an non-human animal with a NK3 R agonist treating, c) measuring the activity of the treated non-human animal, and d) correlating the effect of the antagonist of interest on the activity of the treated non-human animal with the in vivo potency of the antagonist.

### PRIORITY TO RELATED APPLICATIONS

This application claims the benefit of European Application No. 05107892.1, filed Aug. 29, 2005, which is hereby incorporated by reference in its entirety.

### FIELD OF THE INVENTION

The present invention relates generally to methods of assessing in vivo potency of a NK3 receptor antagonist.

### BACKGROUND OF THE INVENTION

Tachykinins are a family of small peptides, e.g. Substance P, Neurokinin A and Neurokinin B, characterized by the shared presence of a common C-terminal amidated sequence Phe-Xaa-Gly-Leu-Met-NH2 (SEQ. ID. NO: 1). Their biological effects are mediated through three distinct types of receptors e.g. Neurokinin 1 (NK1), Neurokinin 2 (NK2) and Neurokinin 3 (NK3), respectively. Tachykinins and their receptors have been the focus of intense research over the past few years. Recently, the selective NK3-receptor (NK3-R) antagonist SR142801 (Osanetant) was shown to be active in a double blind placebo controlled clinical trial in schizophrenia patients (Meltzer H. Y., Arvanitis L., Bauer D., Rein W.: A placebo-controlled evaluation of four novel compounds for the treatment of schizophrenia and schizoaffective disorder. Am J Psych 161:975-84, 2004). However, the pharmacology and function of NK3 receptors in brain and behaviour and animal models are less well studied. There is a need for accurate means to assess the effect of NK3-R in vivo.

### SUMMARY OF THE INVENTION

Therefore, the present invention provides a method of assessing the in vivo potency of a NK3 receptor antagonist comprising:

a) administering to a non-human animal the NK3-R antagonist of interest,

b) administering to non-human animal a NK3-R agonist,

c) measuring the locomotor activity of the treated non-human animal and,

d) correlating the effect of the antagonist of interest on the locomotor activity of said non-human animal with the in vivo potency of the antagonist.

Preferably, said correlation on the induced (agonist) step (d) refers to a comparison of total distance traveled by the non-human animal over a set period of time as against the attenuated (inhibitory effect) antagonist effects on the total distance traveled over a said set period of time.

### DESCRIPTION OF THE FIGURES

FIG. 1 shows a graphical presentation of the locomotor activity induced by increasing doses of N-(3-carboxy-1-oxopropyl)-L-α-aspartyl-L-phenylalanyl-N-methyl-L-phenylalanylglycyl-L-leucyl-L-methioninamide (=senktide).

***=p<0.001 (Dunnet multiple comparison test) versus vehicle.

FIG. 2 shows a graphical presentation of the effect of N-[1-[3-[(3R)-(1-benzoyl-3-(3,4-dichlorophenyl)-3-piperidinyl]propyl]-4-phenyl-4-piperidinyl]-N-methyl-acetamide (10 or 30 mg/kg, IP), 3-hydroxy-2-phenyl-N-[(1S)-1-phenylpropyl]-4-quinolinecarboxamide (10 or 30 mg/kg, IP) and 3-methyl-2-phenyl-N-[(1S)-1-phenylpropyl]-4-quinolinecarboxamide (10 or 30 mg/kg, IP) on locomotor activity induced by N-(3-carboxy-1-oxopropyl)-L-α-aspartyl-L-phenylalanyl-N-methyl-L-phenylalanylglycyl-L-leucyl-L-methioninamide.

#=p<0.05, ###=p<0.001 (un-paired T-test) versus senktide.

### DETAILED DESCRIPTION OF THE INVENTION

Definitions

The term “in vivo potency” as used herein refers to the capacity of a compound, in this case a NK3-R antagonist, to produce the desired effect in a non-human animal, thereby attenuating the effect of NK3 or an NK3-R agonist to the NK3-R is the desired effect.

The term “NK3-R antagonist” or “antagonist” as used herein, refers to a compound capable of attenuating the effect of NK3 or an NK3-R agonist to the NK3-R.

The term “NK3-R agonist” or “agonist” as used herein refers to a compound capable of stimulating the Neurokinin 3 receptor.

The term “locomotor activity” of the non-human includes but is not limited to walking, running, swimming or stereotypic activity such as e.g. grooming, licking or rearing. Preferably, the locomotor activity is measured by determining the distance covered by the non-human animal.

### DETAILED DESCRIPTION

Therefore, the present invention provides a method of assessing the in vivo potency of a NK3 receptor antagonist comprising:

a) administering to (treating) a non-human animal the NK3-R antagonist of interest,

b) administering to (treating) said non-human animal a NK3-R agonist,

c) measuring the locomotor activity of the treated non-human animal and,

d) correlating the effect of the antagonist of interest on the locomotor activity of said non-human animal with the in vivo potency of the antagonist.

Preferably, said correlation on the induced (agonist) step (d) refers to a comparison of total distance traveled by the non-human animal over a set period of time as against the attenuated (inhibitory effect) antagonist effects on the total distance traveled over said set period of time.

The NK3 antagonist may be administered to the non-human animal by known enteral or parenteral routes, including but not limited to oral administration (such as oral gavage, sublingual administration or rectal administration), injection directly into the blood stream (such as intravenous or intra-arterial administration), or various parenteral routes (such as intraperitoneal and subcutaneous routes such as intramuscular), respiratory-based administration via an aerosol, and administration under the skin (i.e., transdermal, transcutaneous or percutaneous), as well as topical administration of the formulated compound of interest. A preferred form of administration of the antagonist is the oral (PO) administration or the intraperitoneal (IP) administration. Preferably, the antagonist is administered at a single dose.

The antagonist is typically administered some time prior to the testing of the activity of the non-human animal. The length of time depends upon the time a compound needs for absorption and to overcome the blood-brain barrier. Preferably, the antagonist is administered to the non-human animal at about 15 to about 240 minutes prior to the testing.

The NK3 receptor agonist may be any agonist of the Neurokinin 3 receptor, preferably the agonist is N-(3-carboxy-1-oxopropyl)-L-α-aspartyl-L-phenylalanyl-N-methyl-L-phenylalanylglycyl-L-leucyl-L-methioninamide (Senktide). The agonist may be administered to the non-human animal by injection into the brain of the animal. More preferably, the agonist is injected into one or more ventricles. More preferably, the agonist is injected into the third (ventral) ventricle of the non-human animal. The agonist is typically administered at a single dose shortly prior to the testing of the activity of the non-human animal. For the injection of the NK3-receptor agonist into the brain, the non-human animal usually is anaestesized. Preferably, the testing starts right after the non-human animal has awakened, which preferably may be about 1 to about 4 minutes after the administration of the NK3-R agonist.

The non-human animal may be any animal excluding human. Preferably, the non-human animal is a rodent, more preferably a gerbil or a mouse. The most preferred non-human animal is a Mongolian gerbil (meriones unguiculates). The non-human animal may also be a non-human animal transgenic for the human NK3-receptor or the gerbil NK3-receptor.

Having now generally described this invention, the same will become better understood by reference to the specific examples, which are included herein for purpose of illustration only and are not intended to be limiting unless otherwise specified, in connection with the following figures.

### EXAMPLE

Commercially available reagents referred to in the example were used according to manufacturer's instructions unless otherwise indicated.

Materials and Methods

Animals and Housing

Male and female Mongolian gerbils [Biological Research Laboratories, 4414 Füllinsdorf, Switzerland] weighing between 40-70 g were used. Gerbils were housed four per cage for approximately four to seven days following arrival in the laboratory colony. The gerbils had ad libitum access to tap water and lab chow in the home cage. The animal quarters were maintained on a 12:12 hour light-dark cycle with light onset at 6 a.m. and with both temperature (21-23° C.) and humidity (55-65%) regulated. All testing was conducted during the light phase of the light-dark cycle. All procedures described here received prior approval from the City of Basel Cantonal Animal Protection Committee based on adherence to federal and local regulations. The experiments were carried out in an animal colony supervised by the veterinary staff of Hoffmann-La Roche Ltd.

Compounds

Angonist:
N-(3-carboxy-1-oxopropyl)-L-α-aspartyl-L-phenylalanyl-N-methyl-L-phenylalanylglycyl-L-leucyl-L-methioninamide (Senktide)

Antagonists:
N-[1-[3-[(3R)-(1-benzoyl-3-(3,4-dichlorophenyl)-3-piperidinyl]propyl]-4-phenyl-4-piperidinyl]-N-methyl-acetamide (SR142801)
3-methyl-2-phenyl-N-[(1S)-1-phenylpropyl]-4-quinolinecarboxamide (SB 222200)
3-hydroxy-2-phenyl-N-[(1S)-1-phenylpropyl]-4-quinolinecarboxamide (Talnetant)
   Preparation of Drugs

The antagonists were prepared immediately prior to use (vehicle 0.3% Tween-80/saline). The antagonistic substances were administered IP or PO, 30 min to 240 min prior to testing. Senktide was dissolved in 0.1% BSA in distilled water and frozen (−20° C.) in aliquots until use, where it was given by direct intracerebroventricular (i.c.v., ICV) injection.

Inhibition of Senktide-Induced Locomotor Activity in Gerbils

Gerbils were anaesthetised by inhalation of isoflurane/oxygen mixture, the scalp was exposed and Senktide (0.01-0.1 nmol/5 μl) was administered into the third (ventral) ventricle (icv) via a cuffed 25 G needle vertically inserted to a depth of 4.5 mm below bregma. The incision was closed using a clip suture and gerbils were placed into perspex boxes on recovery of their righting reflex and then placed into locomotor activity boxes (Accuscan instruments, USA). Activity was then measured automatically for a period of 90 minutes.

Statistical Analysis

Data were analysed using a One way anova followed by Dunnet multiple comparison test or t-test (one-tailed) where appropriate (Statistica, Statsoft).

Results

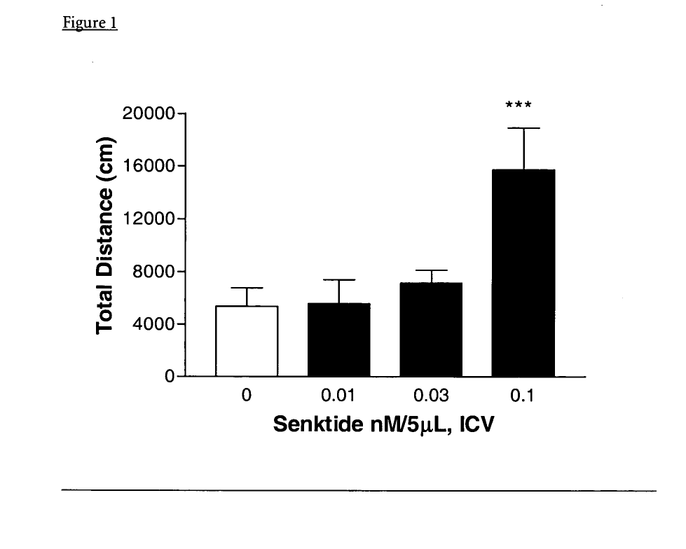

Senktide (0.01, 0.03 or 0.1 nM/5 μL, ICV) induced a significant increase of total distance traveled at a dose of 0.1 nM/5 μL (ICV; FIG. 1).

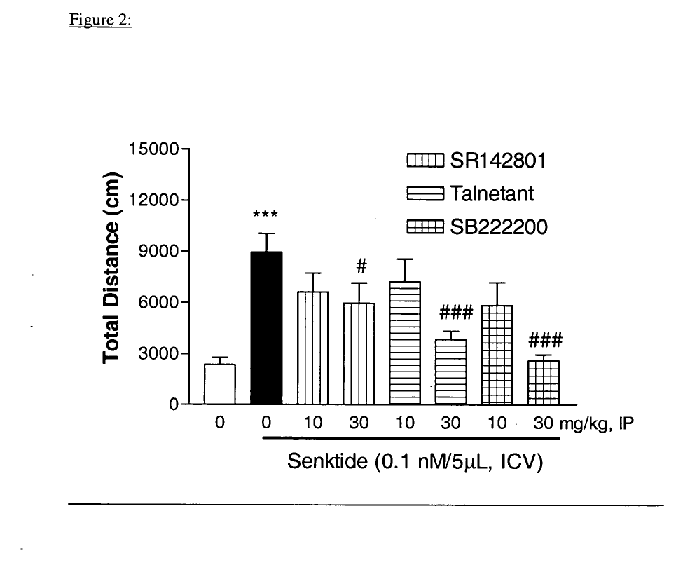

SR142801 (10 or 30 mg/kg IP, 30 min pre-treatment time) significantly reduced senktide (0.1 nM/5 μL, ICV)-induced locomotor activity at a dose of 30 mg/kg (FIG. 2).

Talnetant (10 or 30 mg/kg IP, 30 min pre-treatment time) significantly reduced senktide (0.1 nM/5 μL, ICV)-induced locomotor activity at a dose of 30 mg/kg (FIG. 2).

SB222200 (10 or 30 mg/kg IP, 30 min pre-treatment time) significantly reduced senktide (0.1 nM/5 μL, ICV)-induced locomotor activity at a dose of 30 mg/kg (FIG. 2).

Senktide induced a significant increase in locomotor activity as measured by means of total distance traveled over a period of 90 minutes following the ICV injection. This effect was inhibited by selective NK3 receptor antagonists such as SR142801, Talnetant and SB222200. These data indicate that senktide-induced locomotor activity is a selective NK3 receptor mediated effect and can be used to assess in vivo potency of NK3 receptor antagonists.

1. An method of assessing the in vivo potency of a NK3 receptor antagonist comprising:
a) administering to a non-human animal the NK3 receptor antagonist of interest,
b) administering to said the non-human animal a NK3 receptor agonist,
c) measuring the locomotor activity of said non-human animal and,
d) correlating the effect of the antagonist of interest on the locomotor activity of said non-human animal with the in vivo potency of the antagonist.

2. The method according to claim 1 wherein the NK3-R agonist is N-(3-carboxy-1-oxopropyl)-L-α-aspartyl-L-phenylalanyl-N-methyl-L-phenylalanylglycyl-L-leucyl-L-methioninamide.

3. The method according to claim 1 wherein the agonist is injected into the brain of the non-human animal.

4. The method according to claim 1 wherein the agonist is injected into one or more ventricles.

5. The method according to claim 4 wherein the agonist is injected into the third (ventral) ventricle.

6. The method of claim 1 wherein the non-human animal is a rodent.
